(19) United States
(12) Patent Application Publication
(10) Pub. No.: US 2015/0204835 A1
(43) Pub. Date: Jul. 23, 2015

(54) CHEMICAL SENSORS AND METHODS OF MAKING AND USING THE SAME

(71) Applicants: Liangming Wei, Shanghai (CN); Yafei Zhang, Shanghai (CN)

(72) Inventors: Liangming Wei, Shanghai (CN); Yafei Zhang, Shanghai (CN)

(73) Assignee: Empire Technology Development LLC, Wilmington, DE (US)

(21) Appl. No.: 14/410,902

(22) PCT Filed: Jun. 27, 2012
(86) PCT No.: PCT/CN2012/077620

§ 371 Date: Dec. 23, 2014

(51) Int. Cl. G01N 33/18

(52) U.S. Cl. CPC G01N 33/1813

(57) ABSTRACT

The present application relates to methods of making a chemical sensor including dispersing mesoporous silica structures, an organic solvent, and water to form a composition; and combining one or more chemical sensing molecules with the composition. In some embodiments, the composition includes not more than about 0.6 g of water relative to about 1 g of the mesoporous silica structures. In some embodiments, the chemical sensing molecules include a silane coupling group coupled to a chemical sensing group. Also discloses herein are chemical sensors and methods of using the chemical sensors. The chemical sensors may, in some embodiments, exhibit superior detection of one or more analytes.

### BACKGROUND

Fluorescence chemosensors are sensors that exhibit a change in fluorescence in the presence of an analyte. As such, fluorescence chemosensors can be used to detect an analyte within a composition. As an example, metal ions can be detected using fluorescence chemosensors that exhibit a decrease in fluorescence when exposed to metal ions in a liquid. These chemosensors may therefore be used, for example, to detect impurities in water supplies. The fluorescence chemosensors typically include chemical sensing molecules grafted to a substrate.

### SUMMARY

The foregoing summary is illustrative only and is not intended to be in any way limiting. In addition to the illustrative aspects, embodiments, and features described above, further aspects, embodiments, and features will become apparent by reference to the drawings and the following detailed description.

Some embodiments disclosed herein include a method of making a chemical sensor, the method can include: dispersing mesoporous silica structures, an organic solvent, and water to form a composition, providing one or more chemical sensing molecules comprising a silane coupling group coupled to a chemical sensing group; and combining the chemical sensing molecules with the composition. The composition can include not more than about 0.6 g of water relative to about 1 g of the mesoporous silica structures.

Some embodiments disclosed herein include a chemical sensor prepared according to any of the methods disclosed in the present application.

Some embodiments disclosed herein include a chemical sensor including: one or more mesoporous silica structures having a surface area of at least about 200 m²/g; and one or more chemical sensing molecules including one or more silane coupling groups. The chemical sensing molecules can be coupled to a surface of the mesoporous silica structures and at least a portion of the chemical sensing molecules are crosslinked.

Some embodiments disclosed herein include a method for sensing an analyte in a sample, the method including contacting the sample with a chemical sensor; exposing the chemical sensor to a radiation effective to produce fluorescence from at least one of the chemical sensing molecules; and measuring the amount of fluorescence produced by the chemical sensor. The chemical sensor can include one or more mesoporous silica structures which can have a surface area of at least about 200 m²/g, and one or more chemical sensing molecules comprising one or more silane coupling groups. The chemical sensing molecules can be coupled to a surface of the mesoporous silica structures and at least a portion of the chemical sensing molecules are crosslinked.

Some embodiments disclosed herein include an apparatus for sensing an analyte in a sample, the apparatus including a chemical sensor; at least one light source configured to expose the chemical sensor to a radiation effective to cause at least one of the chemical sensing molecules to emit fluorescence; and at least one light detector configured to measure the emitted fluorescence produced by the chemical sensor. The chemical sensor can include one or more mesoporous silica structures which can have a surface area of at least about 200 m²/g, and one or more chemical sensing molecules comprising one or more silane coupling groups. The chemical sensing molecules can be coupled to a surface of the mesoporous silica structures and at least a portion of the chemical sensing molecules are crosslinked.

Some embodiments disclosed herein include a kit including: a chemical sensor configured to detect an analyte, and one or more positive control samples comprising the analyte. The chemical sensor can include one or more mesoporous silica structures having a surface area of at least about 200 m²/g; and one or more chemical sensing molecules including one or more silane coupling groups. The chemical sensing molecules can be coupled to a surface of the mesoporous silica structures and at least a portion of the chemical sensing molecules are crosslinked.

### BRIEF DESCRIPTION OF THE DRAWINGS

The foregoing and other features of the present disclosure will become more fully apparent from the following description and appended claims, taken in conjunction with the accompanying drawings. Understanding that these drawings depict only several embodiments in accordance with the disclosure and are not to be considered limiting of its scope, the disclosure will be described with additional specificity and detail through use of the accompanying drawings.

FIG. 1 is a flow diagram that depicts some embodiments of a method of making a chemical sensor that is within the scope of the present application.

FIG. 2(a) shows an example of a generic chemical sensing molecule coupled to the surface of silica.

FIG. 2(b) shows an example of two generic chemical sensing molecules coupled to the surface of silica. The chemical sensing molecules are crosslinked.

FIG. 2(c) shows an example of three generic chemical sensing molecules coupled to the surface of silica. The chemical sensing molecules are crosslinked.

FIG. 3 depicts an illustrative embodiment of an apparatus for sensing an analyte that is within the scope of the present application (not to scale).

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be used, and other changes may be made, without departing from the spirit or scope of the subject matter presented here. It will be readily understood that the aspects of the present disclosure, as generally described herein, and illustrated in the Figures, can be arranged, substituted, combined, and designed in a wide variety of different configurations, all of which are explicitly contemplated and make part of this disclosure.

Disclosed herein are methods of making a chemical sensor including dispersing mesoporous silica structures, an organic solvent, and water to form a composition; and combining one or more chemical sensing molecules with the composition. In some embodiments, the composition includes not more than about 0.6 g of water relative to about 1 g of the mesoporous silica structures. In some embodiments, the chemical sensing molecules include a silane coupling group coupled to a chemical sensing group. Also discloses herein are chemical sensors and methods of using the chemical sensors. The chemical sensors may, in some embodiments, exhibit superior detection of one or more analytes.

### DEFINITIONS

As used herein, “alkyl” refers to a straight or branched hydrocarbon chain that comprises a fully saturated (no double or triple bonds) hydrocarbon group. The alkyl group of the compounds may be designated as “C₁-C₄ alkyl” or similar designations. By way of example only, “C₁-C₄ alkyl” indicates that there are one to four carbon atoms in the alkyl chain, i.e., the alkyl chain is selected from methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl, hexyl, and the like.

As used herein, the term “hydroxyl” refers to a —OH group.

As used herein, the term “silanol” refers to a —Si—OH group.

As used herein, the term “acyloxy” refers to —OC(O)-alkyl. In some embodiments, acyloxy can be —OC(O)—C₁₋₆ alkyl. Non-limiting examples of acyloxy substituents include acetoxy, propionyloxy, butyryloxy, and the like.

As used herein, the term “alkoxy” refers to —O-alkyl. In some embodiments, alkoxy can be —O—C₁₋₆ alkyl (or referred to as “C₁₋₆ alkoxy”). Non-limiting examples of alkoxy substituents include methoxy, ethoxy, propoxy, isopropoxy, butoxy, isobutoxy, and the like.

### Method of Making Chemical Sensors

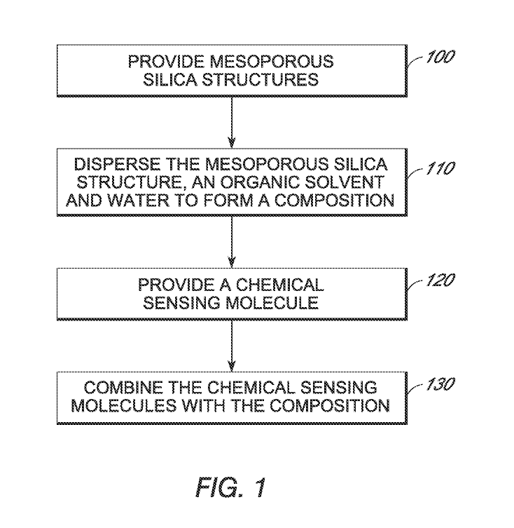

Some embodiments disclosed herein include a method of making a chemical sensor. FIG. 1 is a flow diagram that depicts some embodiments of a method of making a chemical sensor. The method of making the composite may include: an operation “Provide mesoporous silica structures,” illustrated in block 100, an operation “Disperse the mesoporous silica structures, an organic solvent, and water to form a composition,” illustrated in block 110, an operation “Provide a chemical sensing molecule,” illustrated in block 120, and an operation “Combine the chemical sensing molecule with the composition,” illustrated in block 130. Although operations 100, 110, 120, and 130 may be performed sequentially, it will be appreciated that one or more of these operations may be performed at about the same time. These operations may also be performed in a different order than is depicted in FIG. 1.

At operation 100 “Provide mesoporous silica structures,” a mesoporous silica structure is provided for grafting a chemical sensing molecule. The mesoporous silica structures can be one or more of various structures. For example, the mesoporous silica structures can be one of many commercially available mesoporous silica structures, such as MCM-41 type (hexagonal) mesostructured silica available from Sigma-Aldrich. The mesoporous silica structures may, for example, include nanoparticles, an aerogel, a xerogel, a xerogel film or a gel.

The mesoporous silica structures may, in some embodiments, be prepared using conventional sol-gel chemistry techniques. As an example, one or more silicon alkoxides, such as tetraethoxysilane or tetramethoxysilane, can be hydrolyzed and condensed in an aqueous solution to yield colloidal structures. Varying the reaction conditions, such as temperature, pH, and concentration, may affect the final structure of the mesoporous silica. Similarly, surfactants, such as alkylammonium surfactants may be included to further modify the structure. In some embodiments, these surfactants are subsequently removed by, for example, calcination or burning. A non-limiting example of forming a mesoporous silica structure using sol-gel chemistry techniques is described in Metivier, R. et al., J. Mater. Chem., 2005, 15, 2965-2973.

The mesoporous silica structures may have a large surface area. Without being bound to any particular theory, it is believed that a large surface area permits an increased number of chemical sensing molecules to be grafted to the mesoporous silica structures. The greater number of chemical sensing molecules grafted to the mesoporous silica structures may improve the chemical sensing characteristics. The mesoporous silica structure may, for example, have a surface area of at least about 200 m²/g; at least about 300 m²/g; at least about 500 m²/g; at least about 700 m²/g; or at least about 1000 m²/g. The mesoporous silica structure may, for example, have a surface area of less than or equal to about 5000 m²/g; less than or equal to about 3000 m²/g; less than or equal to about 1500 m²/g; or less than or equal to about 1000 m²/g. In some embodiments, the mesoporous silica structures have a surface area from about 200 m²/g to about 5000 m²/g.

The mesoporous silica structures can have various average pore sizes. The mesoporous silica structures may, for example, have an average pore size of less than or equal to about 100 nm; less than or equal to about 75 nm; less than or equal to about 50 nm; or less than or equal to about 30 nm. The mesoporous silica structures may, for example, have an average pore size of at least about 1 nm; at least about 5 nm; at least about 10 nm; or at least about 20 nm. In some embodiments, the mesoporous silica structures have an average pore size from about 1 nm to about 100 nm.

The mesoporous silica structures may be relatively small. For example, the mesoporous silica structures may be nanoparticles having dimensions (e.g., diameter) smaller than about 100 nm. The mesoporous silica structures may, for example, have a largest dimension of less than or equal to about 1 μm; less than or equal to about 500 nm; less than or equal to about 300; or less than or equal to about 100 nm. The mesoporous silica structures may, for example, have a largest dimension of at least about 10 nm; at least about 50 nm; or at least about 100 nm. In some embodiments, the mesoporous silica structures have a largest dimension from about 10 nm to about 1 μm.

The amount of silica in the mesoporous silica structures may vary. The amount of silica in the mesoporous silica structures can be, for example, at least about 50% by weight; at least about 70% by weight; at least about 90% by weight; at least about 95% by weight; at least about 98% by weight; at least about 99% by weight; or at least about 99.5% by weight. The amount of silica in the mesoporous silica structures can be, for example, less than or equal to about 100% by weight; less than or equal to about 99% by weight; less than or equal to about 95% by weight; less than or equal to about 90% by weight; or less than or equal to about 80% by weight. In some embodiments, the amount of silica in the mesoporous silica structures is from about 50% by weight to about 100% by weight.

The mesoporous silica structures may optionally include metal oxides other than silica. Non-limiting examples of other metal oxides include titanium dioxide, germanium dioxide, vanadium oxide, and zirconium oxide. The other metal oxides may, for example, be included in the mesoporous silica structures by including appropriate reactive monomers when performing sol-gel chemistry techniques. Examples of appropriate reactive monomers include, but are not limited to, tetramethoxygermane, tetraisopropoxygermane, tetraethoxygermane, tetrabutoxygermane, aluminum n-butoxide, aluminum isopropoxide, titanium ethoxide, titanium diisopropoxide, titanium methyl phenoxide, vanadium triisopropoxide oxide, vanadium tri-n-propoxide, zirconium n-butoxide, and zirconium n-propoxide.

The amount of other metal oxides within the mesoporous silica structures can vary. The amount of other metal oxides within the mesoporous silica structures may, for example, be less than or equal to about 50% by weight; less than or equal to about 20% by weigh; less than or equal to about 10% by weight; less than or equal to about 5% by weight; or less than or equal to about 1% by weight. The amount of other metal oxides within the mesoporous silica structures may, for example, be at least about 0.5% by weight; at least about 1% by weight; at least about 5% by weight; or at least about 10% by weight. In some embodiments, the amount of other metal oxides within the mesoporous silica structures is from about 0% by weight to about 50% by weight. In some embodiments, the mesoporous silica structures may include no more than trace amounts of other metal oxides.

At operation 110 “Disperse the mesoporous silica structures, an organic solvent, and water to form a composition,” the mesoporous silica structures are combined with an organic solvent and water to form a composition. The method of dispersing the components is not particularly limited and includes various known methods, such as stirring, high-shear mixing, ultrasonication, and the like. As an example, the mesoporous silica structures, the organic solvent, and the water may be vigorously stirred for at least about 1 hour to obtain a mixture. The mesoporous silica structures, the organic solvent, and the water may be dispersed together at about the same time, or these components may be dispersed sequentially. In some embodiments, the dispersing is effective to obtain near-equilibrium conditions in the composition.

The amount of water in the composition may, in some embodiments, be effective to obtain from about 0.1 to about 4 layers of water molecules on the surface of the mesoporous silica structures (assuming that about 5×10¹⁸ water molecules or 83 μmol of water is equivalent to about 1 layer of water molecules on about 1 m² surface of mesoporous silica). The amount of water in the composition may, for example, be effective to obtain at least about 0.1 layers of water molecules; at least about 0.5 layers of water molecules on the surface of the mesoporous silica structures; at least about 1 layers of water molecules on the surface of the mesoporous silica structures; or at least about 1.5 layers of water molecules on the surface of the mesoporous silica structures. The amount of water in the composition may, for example, be effective to obtain less than or equal to about 4 layers of water molecules on the surface of the mesoporous silica structures; less than or equal to about 3 layers of water molecules on the surface of the mesoporous silica structures; or less than or equal to about 2.5 layers of water molecules on the surface of the mesoporous silica structures. As non-limiting examples, the layer can be about 1 water molecule thick, about 2 water molecules thick or about 3 water molecules thick. Without being bound to any particular theory, it is believed that forming a layer of water molecules having the above-described thicknesses improves grafting of chemical sensing molecules to the surface of the mesoporous silica (e.g., improves grafting that may occur in operation 130 of FIG. 1).

As will be appreciated by the skilled artisan, guided by the teachings of the present application, the amount of water used to form the layer of water molecules can vary with the surface area of the mesoporous silica structures. As an example, a composition including about 1 g of mesoporous silica structures having a surface area of about 1000 m²/g has a total silica surface area about 1000 m². Assuming that about 83 μmol of water forms a layer of about 1 water molecule thick, then about 166 μmol of water can be included in the composition to obtain a layer of about 2 water molecules thick. As another example, a composition including about 1 g of mesoporous silica structures having a surface area of about 500 m²/g can include about 83 μmol of water to obtain a layer of about 2 water molecules thick.

The amount of water in the composition may, in some embodiments, be relative to amount of surface area of the mesoporous silica structures. In some embodiments, the amount of water in the composition may be at least about 0.15 g of water relative to an amount of mesoporous silica structures equivalent to about 1000 m² of surface area. In some embodiments, the amount of water in the composition may be at least about 1.5 g of water relative to an amount of mesoporous silica structures equivalent to about 1000 m² of surface area. In some embodiments, the amount of water in the composition may be at least about 2 g of water relative to an amount of mesoporous silica structures equivalent to about 1000 m² of surface area. In some embodiments, the amount of water in the composition may be less than or equal to about 6 g of water relative to an amount of mesoporous silica structures equivalent to about 1000 m² of surface area. In some embodiments, the amount of water in the composition may be less than or equal to about 4.5 g of water relative to an amount of mesoporous silica structures equivalent to about 1000 m² of surface area. In some embodiments, the amount of water in the composition may be less than or equal to about 4 g of water relative to an amount of mesoporous silica structures equivalent to about 1000 m² of surface area. In some embodiments, the amount of water in the composition may be from about 0.15 g to about 6 g of water relative to an amount of mesoporous silica structures equivalent to about 1000 m² of surface area.

As used herein, “an amount of mesoporous silica structures equivalent to about 1000 m² of surface area” is an amount of mesoporous silica structures by weight that contains about 1000 m² of surface area. As one example, 2 g of mesoporous silica structures having a surface area of about 500 m²/g would be an amount of mesoporous silica structures equivalent to about 1000 m² of surface area. As another example, 4 g of mesoporous silica structures having a surface area of about 250 m²/g would be an amount of mesoporous silica structures equivalent to about 1000 m² of surface area.

The organic solvent is not particularly limited and may be a solvent that does not react with silane coupling agents or silica. The organic solvent may, in some embodiments, be a non-polar solvent. In some embodiments, the organic solvent is aprotic. Non-limiting examples of appropriate solvents include pentane, cyclopentane, hexane, cyclohexane, benzene, toluene, and the like. In some embodiments, the organic solvent comprises toluene. The organic solvent can be a mixture of two or more solvents.

In some embodiments, water is substantially immiscible in the organic solvent. The solubility of water in the organic solvent can be, for example, less than or equal to about 100 mg/L; less than or equal to about 75 mg/L; less than or equal to about 50 mg/L; or less than or equal to about 40 mg/L. In some embodiment, the concentration of water in the composition is greater than the solubility of water in the organic solvent. In some embodiment, the concentration of water in the organic solvent is at least two times greater than the solubility of water in the organic solvent. In some embodiment, the concentration of water in the composition is at least five times greater than the solubility of water in the organic solvent. In some embodiment, the concentration of water in the composition is at least ten times greater than the solubility of water in the organic solvent.

At operation 120 “Provide a chemical sensing molecule,” a chemical sensing molecule is provided for grafting to the mesoporous silica structure. The chemical sensing molecule is not particularly limited and numerous chemical sensing molecules are known in the art.

The chemical sensing molecule may, in some embodiments, be a molecule that exhibits fluorescence. In some embodiments, the chemical sensing molecule exhibits fluorescence in the absence of (or trace amounts of) an analyte. In some embodiments, the chemical sensing molecule exhibits reduced fluorescence intensity in the presence of an analyte.

The chemical sensing molecule may be configured to detect one or more analytes. The analyte may be one or more metal cations, such as a heavy metal cations or a transition metal cations. Non-limiting examples of metal cations include U(II), Hg(II), Cu(II), Cd(II), Zn(II), Cr(VI), Pb(II), Sb(III), and Bi(III). The analyte may be one or more of various fluids, such as oxygen, nitrous dioxide, ammonia, or an organic solvent. The analyte may, in some embodiments, be glucose or ATP (adenosine 5′-triphosphate). In some embodiments, the analyte is a nitroaromatic. Non-limiting examples of nitroaromatics include picric acid, nitrobenzene, dinitrobenzene, nitrotoluene, TNT (3,4,6-trinitrotoluene), DNT (2,4-dinitrotoluene), nitrophenol, 1,3,5-trinitrobenzene (TNB), and 2,6-dinitrobenzonitrile (DNB).

The chemical sensing molecule may include a silane coupling group that can couple to the surface of the mesoporous silica structures. As an example, the silane coupling group may react with a silanol group on the surface of the mesoporous silica structures to covalently attach the chemical sensing molecule to the surface of the mesoporous silica structures. In some embodiments, the silane coupling group may include one, two, or three hydrolyzable groups, such as C₁₋₆alkoxy, acyloxy, amine, or chlorine, covalently bonded to a silicon atom. In some embodiments, the silane coupling group can be represented by the formula: —SiR^{A}₃, wherein each R^{A} can independently be hydrogen, C₁₋₆ alkyl, C₁₋₆ alkoxy, or acyloxy, provided that at least one of R^{A} (e.g., one, two, or three) is not hydrogen or C₁₋₆ alkyl. Non-limiting examples of silane coupling groups include trimethoxysilyl, triethoxysilyl, triacetoxysilyl, tripropionyloxysilyl, and the like.

The chemical sensing molecules can, in some embodiments, include a silane coupling agent reacted with a molecule including a chemical sensing functional group. Silane coupling agents can be generally represented by the formula: RₙSiX₍₄₋ₙ₎, where R can be one or more non-hydrolyzable organic groups; X is a hydrolyzable group, such as C₁₋₆alkoxy, acyloxy, amine, or chlorine; and n is 1, 2, or 3. In some embodiments, X is C₁₋₆alkoxy and n is 1. At least one of the non-hydrolyzable groups may have functionality such that the non-hydrolyzable group can couple with (e.g., form a covalent bond with) a molecule containing a chemical sensing functional group. Thus, at least one of the non-hydrolyzable groups may be configured to produce a coupling reaction with the molecule containing a chemical sensing functional group. As an example, an amine-containing non-hydrolyzable group (e.g., as contained in 3-aminopropyltrimethoxysilane) on the silane coupling agent may couple with a carboxylic-acid-containing molecule to form an amide linkage. As another example, an isocyanate-containing non-hydrolyzable group (e.g., as contained in isocyanatopropyltriethoxysilane) on the silane coupling agent may couple with a hydroxyl-containing molecule to form a urethane linkage. A non-limiting example of coupling a silane coupling agent to a molecule including a chemical sensing functional group is described in Rampazzo, E., Journal Materials Chemistry, 2005, 15, 2687-2696.

Non-limiting examples of silane coupling agents that may be used to obtain the chemical sensing molecules include N-(2-aminoethyl)-3-aminopropyltrimethoxysilane, N-(2-aminoethyl)-3-aminopropylmethyldimethoxysilane, 3-aminopropylmethyldiethoxysilane, 3-aminopropyltriethoxysilane, 3-aminopropyltrimethoxysilane, (N-trimethoxysilylpropyl)polyethyleneimine, trimethoxysilylpropyldiethylenetriamine, 3-chloropropyltrimethoxysilane, 1-trimethoxysilyl-2(p,m-chloromethyl)phenylethane, 2-(3,4-epoxycyclohexyl)ethyltrimethoxysilane, 3-glycidoxypropyltrimethoxysilane, isocyanatopropyltriethoxysilane, bis[3-(triethoxysilyl)propyl]tetrasulfide, 3-mercaptopropylmethyldimethoxysilane, or 3-mercaptopropyltrimethoxysilane.

The molecule including a chemical sensing functional group is also not particularly limited. The molecule may be a derivative of a compound that exhibits chemical sensing functionality. As will be appreciated by the skilled artisan, guided by the teachings of the present application, derivatives may be prepared using routine chemical procedures to enable coupling of a silane coupling agent. As an example, various protecting groups may be introduced to form a derivative that selectively couples with the silane coupling agent. As another example, a reactive functional group can be introduced to form a derivative that will react with the non-hydrolyzable functional group of the silane coupling agent (e.g., a carboxylic-acid-containing group is introduced that will react with an amine-containing non-hydrolyzable group on a silane coupling agent).

Although the chemical sensing molecules may be obtained by reacting a silane coupling agent with a molecule including a chemical sensing functional group, the present application is not limited to chemical sensing molecules prepared by this technique.

Non-limiting examples of appropriate chemical sensing molecules include compounds represented by formulae (I)-(IX), where R is C₁₋₆ alkyl and n is 0, 1, or 2:

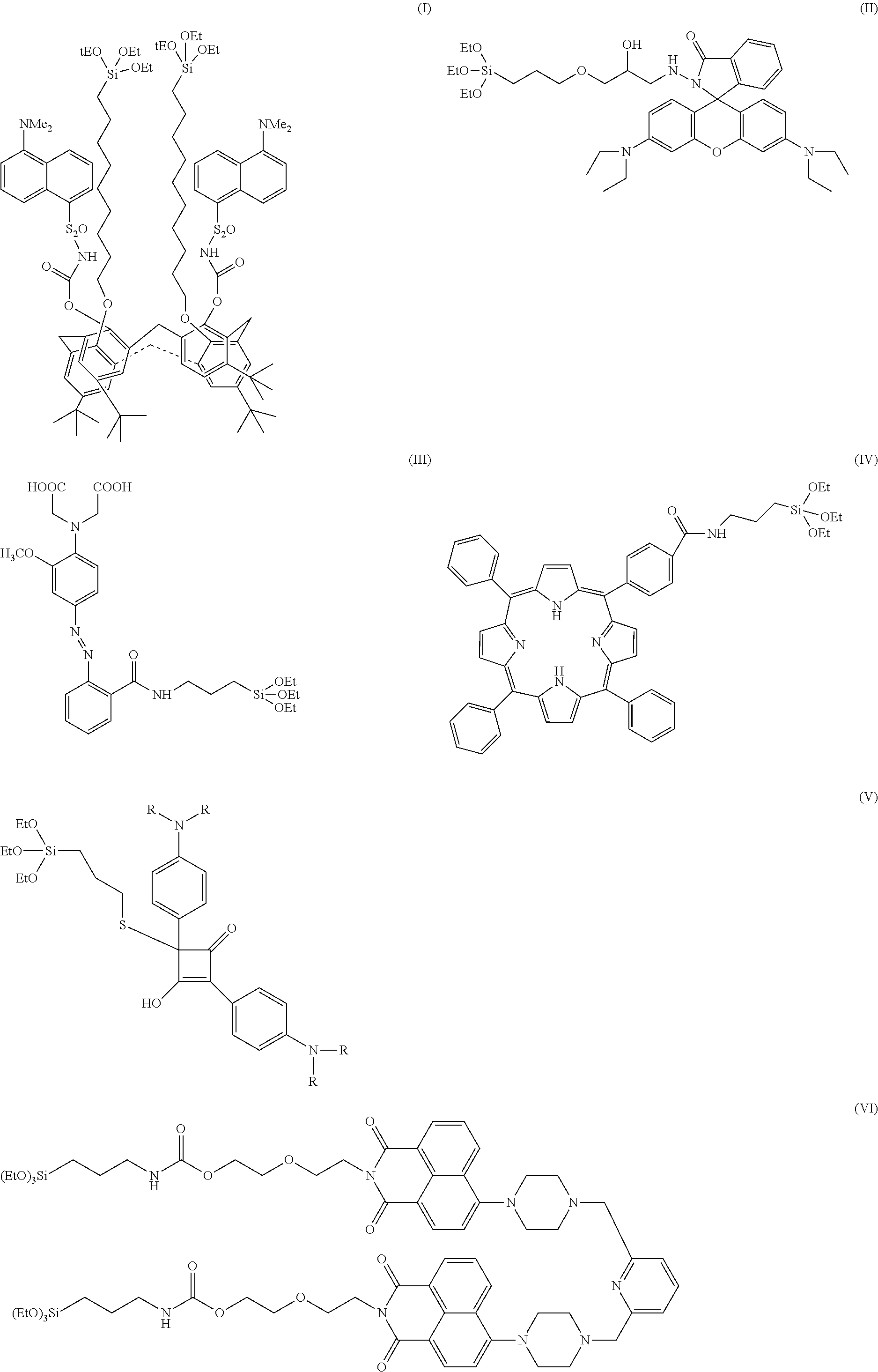

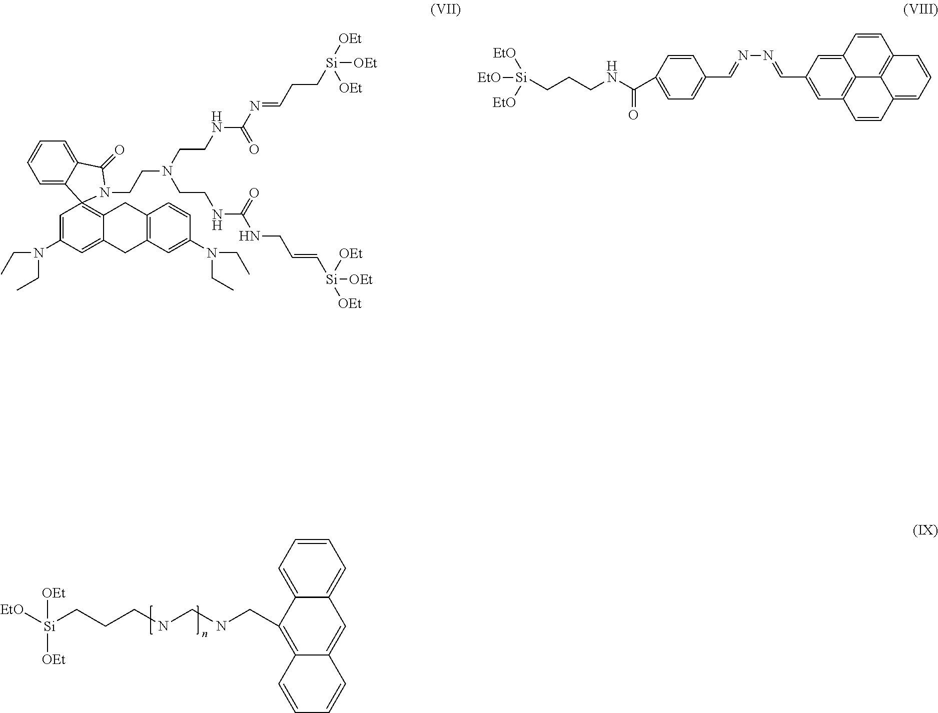

In some embodiments, the chemical sensing molecule is a known chemical sensing molecule. Non-limiting examples of chemical sensing molecules are described in Melde, B. et al., Sensors, 2008, 8, 5202-5228; Metivier, R. et al., J. Mater. Chem., 2005, 15, 2965-2973; Chrisstoffels, L. et al., Angewandte Chemie International, 2000, 39, 2163-2167; Rampazzo, E., Journal Materials Chemistry, 2005, 15, 2687-2696; and Lee, M. et al., Tetrahedron, 2007, 63, 12087-12092.

At operation 130 “Combine the chemical sensing molecules with the composition,” the chemical sensing molecule is combined with the composition. For example, one or more chemical sensing molecules may be dispersed in the composition using standard techniques, such as stirring. In some embodiments, the composition is maintained at conditions effective for the chemical sensing molecules to couple to the surface of the mesoporous silica structures. In some embodiments, the composition is maintained at a temperature from about 20° C. to about 120° C. As an example, the chemical sensing molecules may be combined with the composition and stirred for at least about 1 hour at room temperature to couple the chemical sensing molecules to the mesoporous silica structures. An acid or a base may optionally be combined with the composition to catalyze the coupling reaction.

The amount of the chemical sensing molecule combined with the composition is not particularly limited. The amount of chemical sensing molecules may, for example, be varied according to the desired amount of chemical sensing molecules. In some embodiments, the number of hydrolyzable groups on the silane coupling groups in the chemical sensing molecule multiplied by the molar amount of chemical sensing groups combined with the composition is greater than or about equal to the molar amount of water in the composition. The amount of chemical sensing molecules may also be varied to adjust the density of chemical sensing molecules coupled to the surface of the mesoporous silica structures. Without being bound to any particular theory, it is believed that the silane coupling groups are hydrolyzed by water in the composition before coupling with the mesoporous silica structures.

The mesoporous silica structures may optionally be isolated from the composition using standard methods after coupling the chemical sensing molecules. As an example, the composition can be filtered and dried to isolate the mesoporous silica structure. The dried mesoporous silica structures may optionally be further packaged in a hermetically sealed container for subsequent use.

### Chemical Sensors

Some embodiments disclosed herein include a chemical sensor. The chemical sensor may include one or more mesoporous silica structures and one or more chemical sensing molecules including a silane coupling group. The chemical sensing molecules may be coupled to a surface of the mesoporous silica structures. The chemical sensors may, for example, be prepared by the methods disclosed in the present application.

The mesoporous silica structures are not particularly limited and may include any of the characteristics described with respect to the method of making a chemical sensor. As an example, the mesoporous silica structures may have a surface area from about 200 m²/g to about 5000 m²/g. As another example, the mesoporous silica structures may include nanoparticles, an aerogel, a xerogel, a xerogel film or a gel.

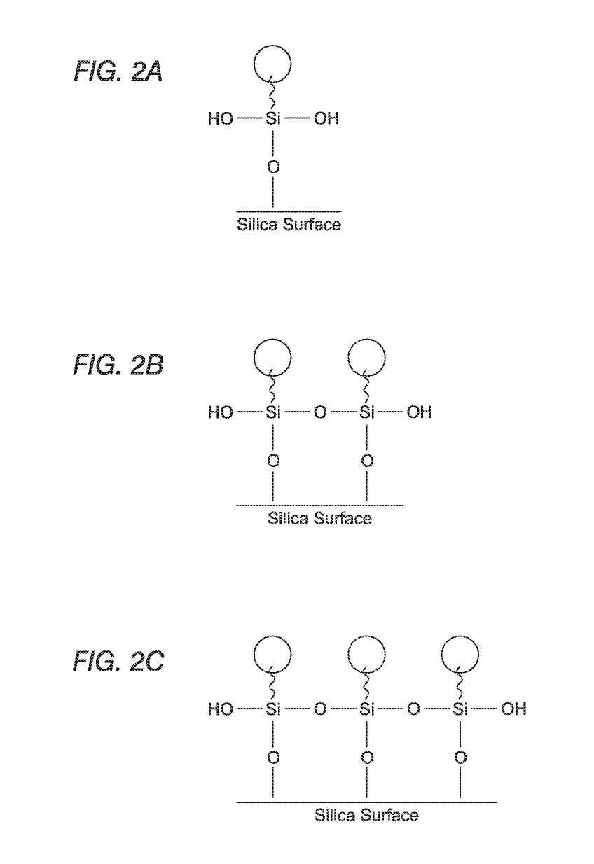

The chemical sensing molecules are also not particularly limited and may include any of the chemical sensing molecules described with respect to the method of making a chemical sensor. For example, the chemical sensing molecules can be at least one of the compounds of formulae (I)-(IX). The chemical sensing molecule can be coupled to a surface of the mesoporous silica structures. In some embodiments, the silane coupling group on the chemical sensing molecule is covalently bonded to the mesoporous silica. Thus, for example, at least one hydrolyzable group on the silane coupling group of the chemical sensing molecule can be hydrolyzed and the silane coupling group may be covalently bonded to an oxygen atom that is bonded to the mesoporous silica. FIG. 2(a) shows one example of a generic chemical sensing molecule coupled to silica.

The chemical sensing molecules, in some embodiments, may be crosslinked on the surface of the mesoporous silica. As used herein, chemical sensing molecules are “crosslinked” when the silane couplings group on a chemical sensing molecule is covalently bonded to a common oxygen atom that is bonded to a silane coupling group of a second chemical sensing molecule. As an example, the two chemical sensing molecules may each have a hydrolyzable group that is hydrolyzed and then the silane coupling groups can be condensated to form a covalent bond with a common oxygen atom. FIG. 2(b) shows one example of two generic chemical sensing molecules that are crosslinked on the surface of the mesoporous silica. FIG. 2(c) shows one example of three generic chemical sensing molecules that are crosslinked on the surface of the mesoporous silica.

The degree of crosslinking between the chemical sensing molecules may vary. In some embodiments, the molar amount of chemical sensing molecules crosslinked to at least one other chemical sensing molecule is at least about 50% by mole of the total molar amount of chemical sensing molecules coupled to the mesoporous silica structures. In some embodiments, the molar amount of chemical sensing molecules crosslinked to at least one other chemical sensing molecule is at least about 80% by mole of the total molar amount of chemical sensing molecules coupled to the mesoporous silica structures. In some embodiments, the molar amount of chemical sensing molecules crosslinked to at least one other chemical sensing molecule is at least about 90% by mole of the total molar amount of chemical sensing molecules coupled to the mesoporous silica structures. In some embodiments, the molar amount of chemical sensing molecules crosslinked to two other chemical sensing molecule is at least about 50% by mole of the total molar amount of chemical sensing molecules coupled to the mesoporous silica structures. In some embodiments, the molar amount of chemical sensing molecules crosslinked to two other chemical sensing molecule is at least about 80% by mole of the total molar amount of chemical sensing molecules coupled to the mesoporous silica structures. In some embodiments, the molar amount of chemical sensing molecules crosslinked to two other chemical sensing molecule is at least about 90% by mole of the total molar amount of chemical sensing molecules coupled to the mesoporous silica structures. The degree of crosslinking may be determined using routine techniques, such as by solid-state nuclear magnetic resonance (NMR).

The chemical sensor may, in some embodiments, have substantially all of the chemical sensing molecules coupled to the surface of the mesoporous silica structures. In contrast, the chemical sensor may have no more than trace amounts of the chemical sensing molecules coupled to internal regions of the mesoporous. The chemical sensors may, for example, be almost exclusively coupled to surfaces of the mesoporous silica structures by grafting the chemical sensing molecules after the mesoporous silica structures are formed (e.g., operation 100 in FIG. 1 is performed before operation 130 in FIG. 1). In contrast, combining the chemical sensing molecules with silica alkoxides during a sol-gel process for making the mesoporous silica structures may result in the chemical sensing molecules being coupled to internal regions of the mesoporous silica structures.

The chemical sensors may have varying densities of chemical sensing molecules on the surface of the mesoporous silica structures. The density may be controlled, for example, by the amount of water or chemical sensing molecules included in the method of making the chemical sensor. The chemical sensor may include, for example, a high density of chemical sensing molecules that can advantageously improve sensing characteristics of the chemical sensor. In some embodiments, at least about 5 μmol of the chemical sensing molecules are coupled to the surface of the mesoporous silica structures relative to an amount of mesoporous silica structures equivalent to about 1 m² of surface area. In some embodiments, at least about 6 μmol of the chemical sensing molecules are coupled to the surface of the mesoporous silica structures relative to an amount of mesoporous silica structures equivalent to about 1 m² of surface area. In some embodiments, at least about 7 μmol of the chemical sensing molecules are coupled to the surface of the mesoporous silica structures relative to an amount of mesoporous silica structures equivalent to about 1 m² of surface area. In some embodiments, at least about 8 μmol of the chemical sensing molecules are coupled to the surface of the mesoporous silica structures relative to an amount of mesoporous silica structures equivalent to about 1 m² of surface area.

The chemical sensor may, in some embodiments, have few silanol groups on the surface of the mesoporous silica structures. Without being bound to any particular theory, it is believed that the chemical sensing molecules can react with silanol groups to couple to the mesoporous silica structures. As such, low molar amounts of silanol groups on the surface of the silica may indicate a dense coverage of chemical sensing molecules. In some embodiments, a molar amount of the chemical sensing molecules coupled to the surface of the mesoporous silica structures relative to a molar amount of silanol groups on the surface of the mesoporous silica structures is at least about 5:1. In some embodiments, a molar amount of the chemical sensing molecules coupled to the surface of the mesoporous silica structures relative to a molar amount of silanol groups on the surface of the mesoporous silica structures is at least about 10:1. In some embodiments, a molar amount of the chemical sensing molecules coupled to the surface of the mesoporous silica structures relative to a molar amount of silanol groups on the surface of the mesoporous silica structures is at least about 20:1.

The chemical sensor may, in some embodiments, exhibit fluorescence. In some embodiments, the chemical sensor exhibits fluorescence in the absence of (or trace amounts of) an analyte. In some embodiments, the chemical sensor exhibits reduced fluorescence intensity in the presence of an analyte.

The chemical sensor may be configured to detect one or more analytes. The analyte may be one or more metal cations, such as heavy metal cations or transition metal cations. Non-limiting examples of metal cations include U(II), Hg(II), Cu(II), Cd(II), Zn(II), Cr(VI), Pb(II), Sb(III), and Bi(III). The analyte may be one or more of various fluids, such as oxygen, nitrous dioxide, ammonia, or an organic solvent. The analyte may, in some embodiments, be glucose or ATP (adenosine 5′-triphosphate). In some embodiments, the analyte is a nitroaromatic. Non-limiting examples of nitroaromatics include picric acid, nitrobenzene, dinitrobenzene, nitrotoluene, TNT (3,4,6-trinitrotoluene), DNT (2,4-dinitrotoluene), nitrophenol, 1,3,5-trinitrobenzene (TNB), and 2,6-dinitrobenzonitrile (DNB).

### Methods, Apparatuses, and Kits for Sensing an Analyte

Some embodiments disclosed herein include a method for sensing an analyte in a sample. The method may include contacting the sample with a chemical sensor, exposing the chemical sensor to a radiation, and measuring the amount of fluorescence produced by the chemical sensor. The chemical sensor can be any of the chemical sensors disclosed in the present application and may include one or more chemical sensing molecules coupled to a mesoporous silica structure. In some embodiment, the radiation applied to the chemical sensor is effective to produce fluorescence from at least one of the chemical sensing molecules. The radiation may, for example, have a wavelength of peak emission from about 200 nm to about 800 nm. The radiation may include at least one of ultraviolet, blue, yellow, green, or red radiation.

The fluorescence exhibited by the chemical sensor may, in some embodiment, decrease in the presence of analyte. Thus, the intensity of fluorescence exhibited by the chemical sensor while contacting a sample may be correlated with the concentration of an analyte in the sample.

The analyte may be any compound that affects the fluorescence properties of the chemical sensor when contacting the chemical sensor. In some embodiments, the analyte decreases the fluorescence intensity of the chemical sensor when contacting the chemical sensor. The analyte may be one or more metal cations, such as heavy metal cations or transition metal cations. Non-limiting examples of metal cations include U(II), Hg(II), Cu(II), Cd(II), Zn(II), Cr(VI), Pb(II), Sb(III), Ag(I), and Bi(III). The analyte may be one or more of various fluids, such as oxygen, nitrous dioxide, ammonia, or an organic solvent. The analyte may, in some embodiments, be glucose or ATP (adenosine 5′-triphosphate). In some embodiments, the analyte is a nitroaromatic. Non-limiting examples of nitroaromatics include picric acid, nitrobenzene, dinitrobenzene, nitrotoluene, TNT (3,4,6-trinitrotoluene), DNT (2,4-dinitrotoluene), nitrophenol, 1,3,5-trinitrobenzene (TNB), and 2,6-dinitrobenzonitrile (DNB).

In some embodiments, the method includes chemical sensor having a chemical sensing molecule represented by formula (I) and the analyte is Hg(II). In some embodiments, the method includes chemical sensor having a chemical sensing molecule represented by formula (II) and the analyte is Hg(II). In some embodiments, the method includes chemical sensor having a chemical sensing molecule represented by formula (III) and the analyte is Hg(II). In some embodiments, the method includes chemical sensor having a chemical sensing molecule represented by formula (IV) and the analyte is Hg(II). In some embodiments, the method includes chemical sensor having a chemical sensing molecule represented by formula (V) and the analyte is Hg(II). In some embodiments, the method includes chemical sensor having a chemical sensing molecule represented by formula (VI) and the analyte is Hg(II) or Hg(I). In some embodiments, the method includes chemical sensor having a chemical sensing molecule represented by formula (VII) and the analyte is Hg(II). In some embodiments, the method includes chemical sensor having a chemical sensing molecule represented by formula (VIII) and the analyte is Hg(II), Pb(II), Cd(II), Cu(II) or Ag(I). In some embodiments, the method includes chemical sensor having a chemical sensing molecule represented by formula (IX) and the analyte is ATP (adenosine 5′-triphosphate).

Some embodiments disclosed herein include an apparatus for sensing an analyte in a sample. The apparatus may include a chemical sensor, at least one light source configured to expose the chemical sensor to a radiation effective to cause the chemical sensor to fluoresce, and at least one light detector configured to measure the emitted fluorescence produced by the chemical sensor. The chemical sensor can be any one of the chemical sensors disclosed in the present application. In some embodiments, the apparatus is configured to perform any one of the methods for sensing an analyte disclosed in the present application.

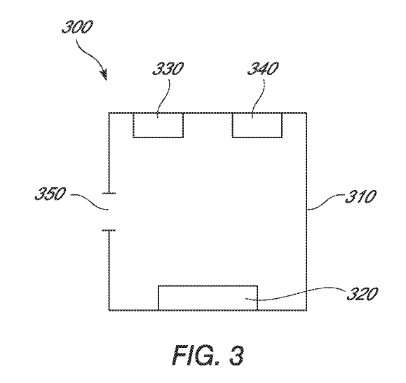

FIG. 3 depicts an illustrative embodiment of an apparatus for sensing an analyte that is within the scope of the present application. Apparatus 300 can include housing 310 that contains composition 320, light source 330, light detector 340, and port 350. Composition 320 can include any of the chemical sensors described in the present application. Light source 330 is configured to emit radiation effective to produce fluorescence from composition 320. For example, light source 330 can be an InGaN semiconductor that emits blue or ultraviolet radiation. Light detector 340 can be configured to measure light emission from composition 320. Port 350 can be configured to receive a sample into the housing. Thus, for example, a sample suspected of containing an analyte may be placed into housing 310 via port 350, so that the sample contacts composition 320. Light source 330 may then emit light and the fluorescence from composition 320 is detected by light detector 340. The amount of fluorescence may then be correlated with the presence of the analyte in the sample.

In some embodiments, the apparatus for sensing an analyte includes a processor coupled to at least the light source and light detector (not shown). The processor may be configured to synchronize both emitting light from the light source and detecting fluorescence with the light detector. The processor may also receive measurement data from the light detector and automatically correlate this data with the presence of an analyte.

Some embodiments disclosed herein include a kit for sensing an analyte. The kit may include a chemical sensor configured to detect an analyte and one or more positive control samples that include the analyte. The chemical sensor can be any one of the chemical sensors disclosed in the present application. In some embodiments, the positive control samples are identified as containing the analyte. In some embodiments, one or more positive control samples include from about 10 μM to about 160 μM Hg(II). In some embodiments, the kit may include a negative control sample that is substantially free of the analyte. In some embodiments, the negative control sample includes less than or equal to about 0.01 μM of Hg(II). The negative control sample may optionally be identified as being free of the analyte.

The kit may optionally include instructions for performing any one of the methods of sensing an analyte in a sample as described in the present application using components in the kit. In some embodiments, the instructions describe a method including contacting the chemical sensor with the sample, exposing the chemical sensor to a radiation effective to produce fluorescence from at least one of the chemical sensing molecules, and measuring the amount of fluorescence produced by the chemical sensor.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

### EXAMPLES

One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

### Example 1

### Preparing Mesoporous Silica Nanoparticles

About 1 g of cetyl ammonium bromide are dissolved in about 480 mL of water and then about 5 g tetraethoxysilane and about 3.5 mL of 2 M NaOH are added to the composition. The composition is maintained at a temperature from about 70° C. to about 90° C. under vigorous stirring conditions (e.g., 400-2000 rpm) for about 4 hours to yield mesoporous silica nanoparticles.

### Example 2

### Grafting Chemical Sensing Molecules

About 1 g of mesoporous silica nanoparticles having a surface area of about 1000 m²/g are intermixed with about 50 mL of toluene and about 0.3 mL of water, and the mixture is stirred for 2-3 hours. About 32.5 mg of the chemical sensing molecule represented by Formula (I) is added to the composition with about 3 mL of 1 M acetic acid solution in water. The composition is stirred and maintained at a temperature from about 20° C. to about 70° C. for about 20 hours. The resulting chemical sensors are filtered and dried from the mixture.

### Example 3

### Using Chemical Sensors to detect Mercury

0.2 mg of the chemical sensors are dispersed in 2.5 mL of water and the fluorescence intensity at about 530 nm is measured using an excitation wavelength of about 350 nm. Hg(II) is added to the composition to obtain a mercury concentration of 80 μM and the fluorescence intensity measurement is repeated. The measured intensity is significantly decreased relative to the first measurement indicating that the chemical sensor can detect mercury in a sample.

### Example 4

### Using Chemical Sensors to Measure Mercury Content in River Water

0.2 mg of chemical sensors containing the chemical sensing molecule represented by Formula VI is dispersed in 10 mL of pure water (negative control sample) and the fluorescence intensity at about 530 nm is measured using an excitation wavelength of about 350 nm. Hg(II) is added to the water to obtain 10 μM, 20 μM, 40 μM, 80 μM, and 160 μM solutions (positive control samples), and the fluorescence intensity measurement is repeated at each concentration. The measured intensity relative to concentration of Hg for the control samples is plotted on a graph. To measure the concentration of Hg (II) in the river water sample, 0.2 mg of the chemical sensors is added into a 10 ml river water sample and the fluorescence intensity is measured. The concentration of Hg (II) is interpolated using the plotted data from the control samples.

1. A method of making a chemical sensor, the method comprising:
dispersing mesoporous silica structures, an organic solvent, and water to form a composition, wherein the composition comprises not more than about 0.6 g of water relative to about 1 g of the mesoporous silica structures;
providing one or more chemical sensing molecules comprising a silane coupling group coupled to a chemical sensing group; and
combining the chemical sensing molecules with the composition.

2. The method of claim 1, wherein dispersing the mesoporous silica structures comprises dispersing mesoporous silica structures have a surface area of at least about 200 m²/g.

3-6. (canceled)

7. The method of claim 1, wherein dispersing the mesoporous silica, the organic solvent, and the water comprises forming a layer of water molecules on the mesoporous silica structures.

8. The method of claim 7, wherein forming the layer of water molecules comprises forming the layer with a thickness of about 0.1 water molecules to about 4 water molecules.

9. The method of claim 1, wherein the composition comprises about 0.015 g to about 0.6 g of water relative to an amount of mesoporous silica structures equivalent to about 1000 m² of surface area.

10. The method of claim 1, wherein the one or more chemical sensing molecules are obtained by reacting a silane coupling agent with a molecule comprising a chemical sensing functional group.

11. The method of claim 10, wherein the silane coupling agent is N-(2-aminoethyl)-3-aminopropyltrimethoxysilane, N-(2-aminoethyl)-3-aminopropylmethyldimethoxysilane, 3-aminopropylmethyldiethoxysilane, 3-aminopropyltriethoxysilane, 3-aminopropyltrimethoxysilane, (N-trimethoxysilylpropyl)polyethyleneimine, trimethoxysilylpropyldiethylenetriamine, 3-chloropropyltrimethoxysilane, 1-trimethoxysilyl-2(p,m-chloromethyl)phenylethane, 2-(3,4-epoxycyclohexyl)ethyltrimethoxysilane, 3-glycidoxypropyltrimethoxysilane, isocyanatopropyltriethoxysilane, bis[3-(triethoxysilyl)propyl]tetrasulfide, 3-mercaptopropylmethyldimethoxysilane, or 3-mercaptopropyltrimethoxysilane.

12. The method of claim 1, wherein at least one of the chemical sensing molecules is a compound represent by one of formulae (I)-(IX): 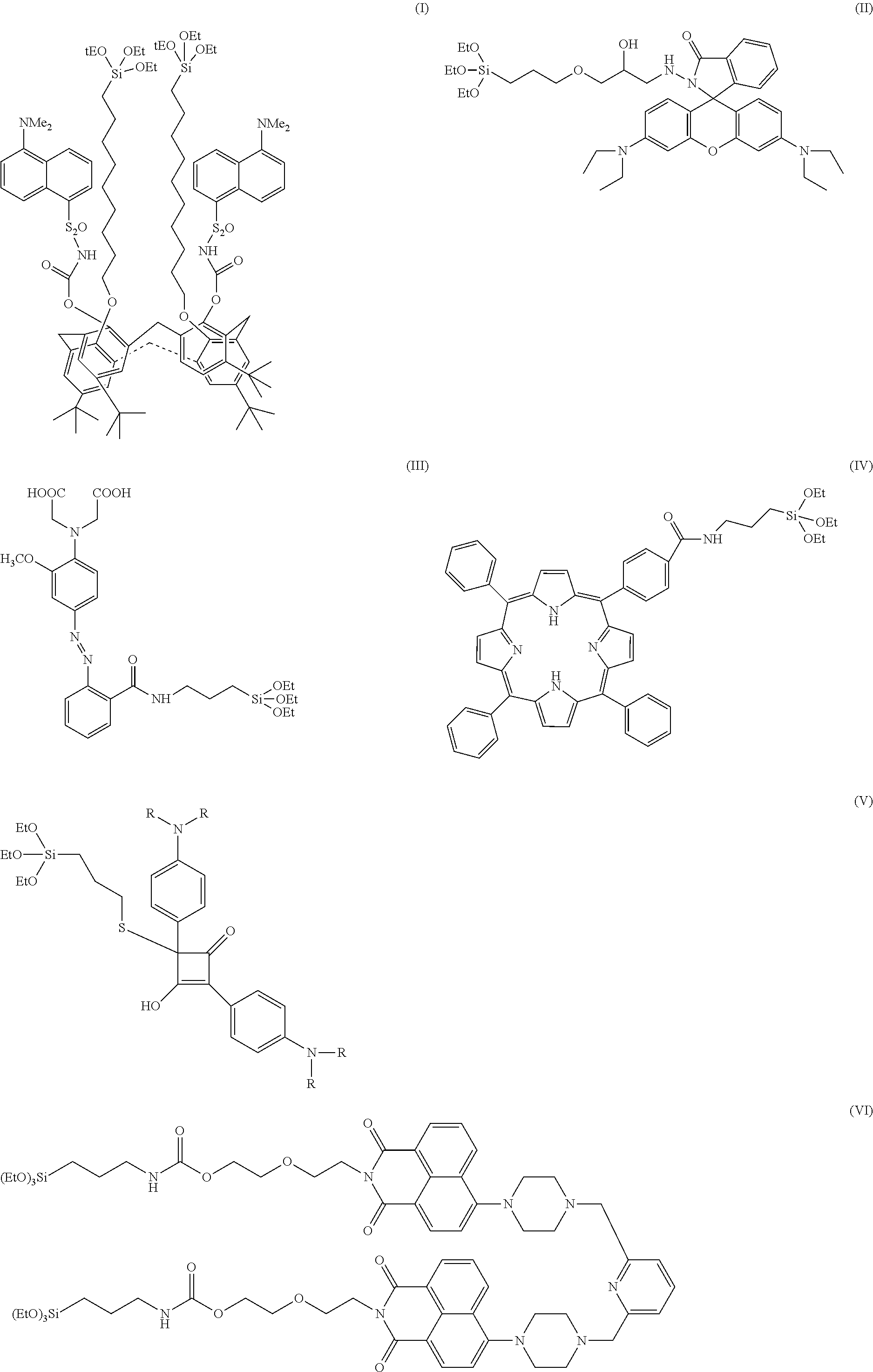 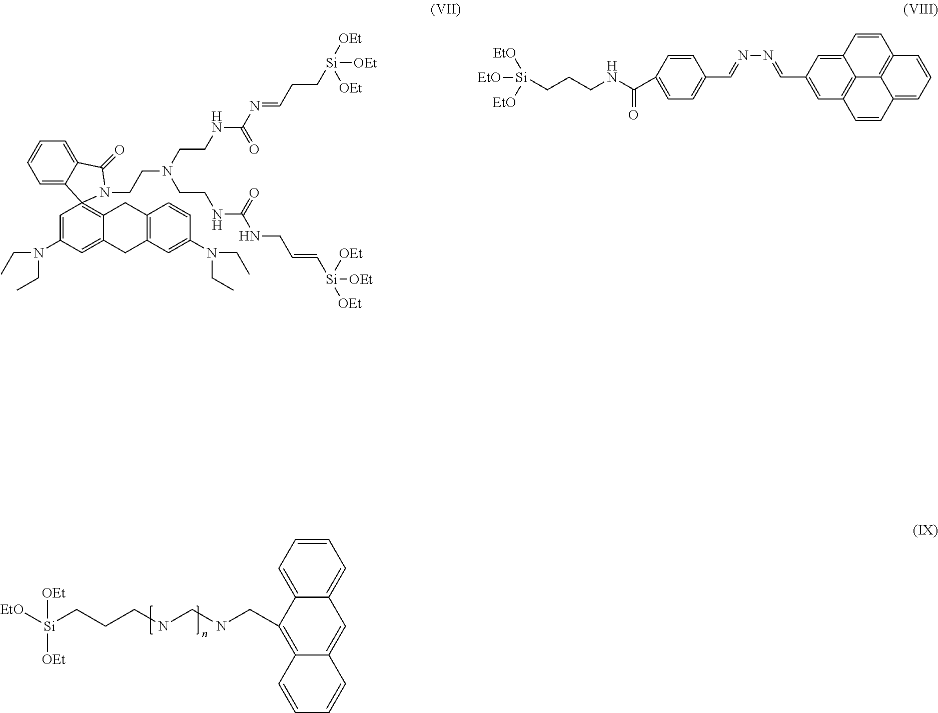
wherein n is 0, 1, or 2, and R is C₁₋₆ alkyl.

13. (canceled)

14. A chemical sensor comprising:
one or more mesoporous silica structures having a surface area of at least about 200 m²/g; and
one or more chemical sensing molecules comprising one or more silane coupling groups, wherein the chemical sensing molecules are coupled to a surface of the mesoporous silica structures and at least a portion of the chemical sensing molecules are crosslinked, and wherein a molar amount of the chemical sensing molecules coupled to the surface of the mesoporous silica structures relative to a molar amount of silanol groups on the surface of the mesoporous silica structures is at least about 5:1.

15. (canceled)

16. The chemical sensor of claim 14, wherein at least about 5 μmol of the chemical sensing molecules are coupled to the surface of the mesoporous silica structures relative to an amount of mesoporous silica structures equivalent to about 1 m² of surface area.

17. The chemical sensor of claim 14, wherein the mesoporous silica structures have an average pore size of less than about 100 nm.

18. The chemical sensor of claim 14, wherein the mesoporous silica structures are nanoparticles, an aerogel, a xerogel film, a xerogel, or a gel.

19. The chemical sensor of claim 14, wherein the mesoporous silica structures have a largest dimension of no more than about 1 μm.

20. The chemical sensor of claim 14, wherein the mesoporous silica structures comprise at least 50% by weight silica.

21. The chemical sensor of claim 14, wherein at least one of the chemical sensing molecules is a compound represent by one of formulae (I)-(IX): 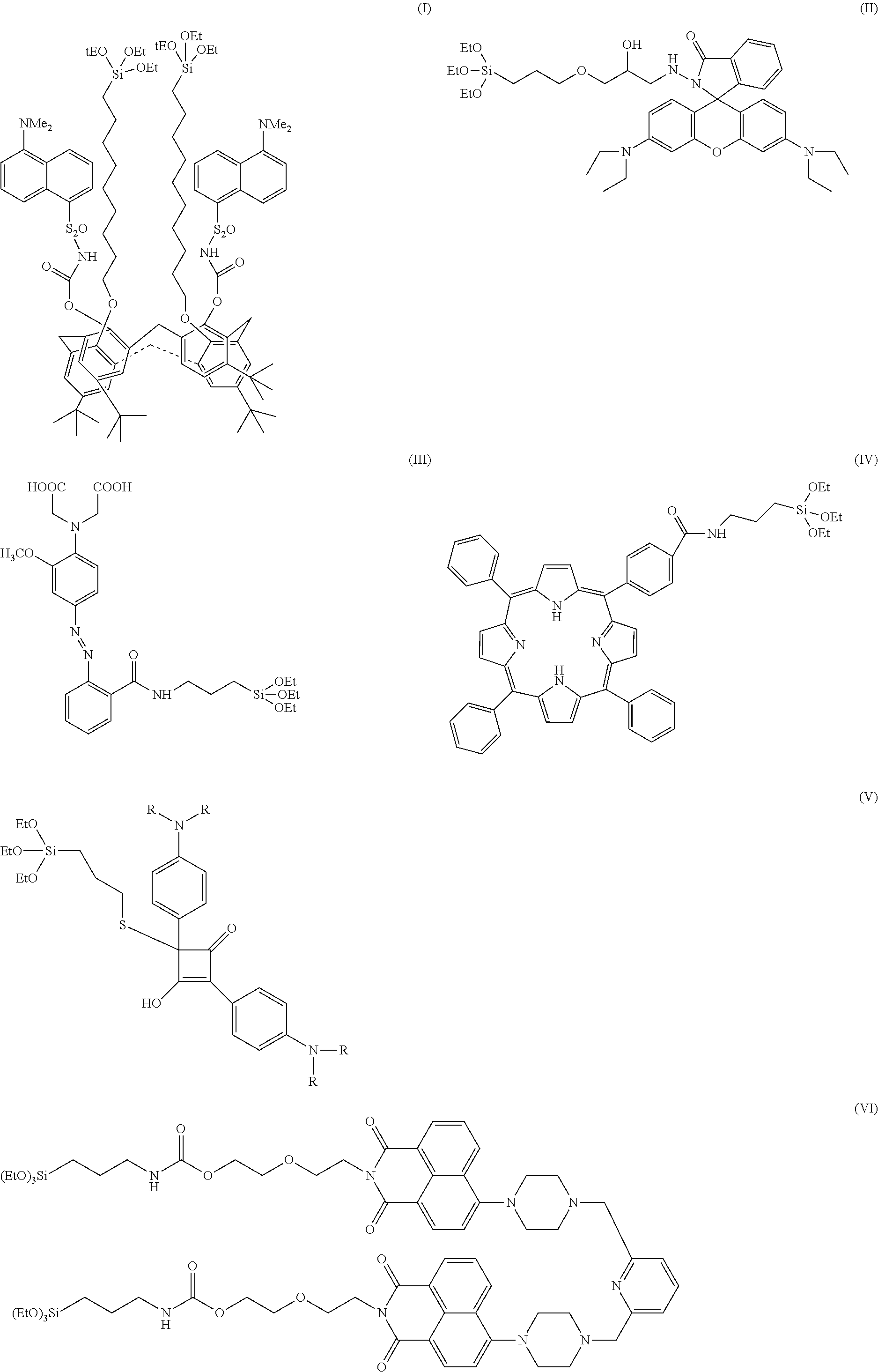 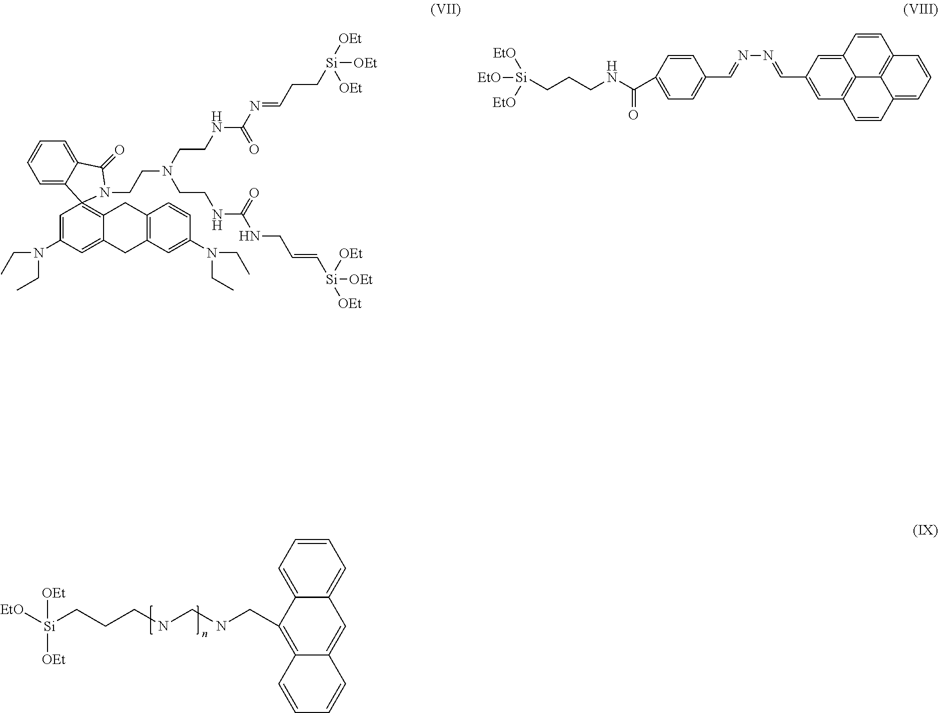
wherein n is 0, 1, or 2, and R is C₁₋₆ alkyl.

22. A method for sensing an analyte in a sample, the method comprising:
contacting the sample with a chemical sensor comprising:
one or more mesoporous silica structures having a surface area of at least about 200 m²/g; and
one or more chemical sensing molecules comprising one or more silane coupling groups, wherein the chemical sensing molecules are coupled to a surface of the mesoporous silica structures and at least a portion of the chemical sensing molecules are crosslinked;
exposing the chemical sensor to a radiation effective to produce fluorescence from at least one of the chemical sensing molecules; and
measuring the amount of fluorescence produced by the chemical sensor.

23. The method of claim 22, wherein the intensity of fluorescence produced by the chemical sensor decreases with an increase of the amount of the analyte in the sample.

24. The method of claim 22, wherein the analyte comprises a metal cation, oxygen, nitrous dioxide, a nitroaromatic, ammonia, an organic solvent, ATP (adenosine 5′-triphosphate), or glucose.

25. The method of claim 22, wherein the analyte comprises U(II), Hg(II), Cu(II), Cd(II), Zn(II), Cr(VI), Pb(II), Sb(III), Ag(I) or Bi(III).

26. The method of claim 22, wherein the analyte comprises picric acid, nitrobenzene, dinitrobenzene, nitrotoluene, TNT (3,4,6-trinitrotoluene), DNT (2,4-dinitrotoluene), nitrophenol, 1,3,5-trinitrobenzene (TNB), and 2,6-dinitrobenzonitrile (DNB).

27. An apparatus for sensing an analyte in a sample, the apparatus comprising:
a chemical sensor comprising:
one or more mesoporous silica structures having a surface area of at least about 200 m²/g; and
one or more chemical sensing molecules comprising one or more silane coupling groups, wherein the chemical sensing molecules are coupled to a surface of the mesoporous silica structures and at least a portion of the chemical sensing molecules are crosslinked;
at least one light source configured to expose the chemical sensor to a radiation effective to cause at least one of the chemical sensing molecules to emit fluorescence;
at least one light detector configured to measure the emitted fluorescence produced by the chemical sensor; and
a processor coupled to at least the light source and light detector, wherein the processor is configured to synchronize emitting radiation from the light source and measuring emitted fluorescence with the light detector.

28. The apparatus of claim 27, further comprising a housing, wherein the housing contains the chemical sensor and is configured to receive the sample.

29. (canceled)

30. The apparatus of claim 27, wherein the processor is further configured to receive measurement data from the light detector and automatically correlate the measurement data with an amount of the analyte in the sample.

31-33. (canceled)
